(19) United States
(12) Patent Application Publication
(10) Pub. No.: US 2010/0291485 A1
(43) Pub. Date: Nov. 18, 2010

(54) NANOSCALE MOLECULE SYNTHESIS

(76) Inventors: Troy Lapsys, Albuquerque, NM (US); Viswanath Krishnamoorthy, Albuquerque, NM (US); Paul Bentley, Albuquerque, NM (US); Uday Khire, Orange, CT (US); Scott Miller, Woodbridge, CT (US); Peter Mikochik, Albuquerque, NM (US)

Correspondence Address: The Grafe Law Office, P.C., P.O. Box 2689, Corrales, NM, 87048, US

(21) Appl. No.: 12/713,148

(22) Filed: Feb. 25, 2010

Related U.S. Application Data
(63) Continuation-in-part of application No. PCT/US2009/056858, filed on Sep. 14, 2009.
(60) Provisional application No. 61/155,302, filed on Feb. 25, 2009.
(60) Provisional application No. 61/237,353, filed on Aug. 27, 2009.
(60) Provisional application No. 61/237,353, filed on Aug. 27, 2009.
(60) Provisional application No. 61/110,535, filed on Oct. 31, 2008.
(60) Provisional application No. 61/096,802, filed on Sep. 14, 2008.

(51) Int. Cl. G03F 7/20; C07H 21/04

(52) U.S. Cl. 430/311; 536/23.1

(57) ABSTRACT

This invention relates to nanoscale molecule synthesis, including three-dimensional addressable arrays of biopolymeric nucleic acids and processes for manufacturing such arrays. Such arrays can be functionalized with complementary chemical reactive probes to provide catalytic enzymes.

### CROSS REFERENCE TO RELATED APPLICATIONS

This application claims priority to, as a continuation of, PCT application PCT/US2009/056858, filed Sep. 14, 2009, which application claimed priority to U.S. provisional applications 61/237353 filed Aug. 27, 2009, 61/110,535 filed Oct. 31, 2008, and 61/096802 filed Sep. 14, 2008; and U.S. provisional applications 61/237353 filed Aug. 27, 2009, 61/110,535 filed Oct. 31, 2008, and 61/096802 filed Sep. 14, 2008; and to U.S. provisional application 61/155,302 filed Feb. 25, 2009, each of which is incorporated herein by reference.

### TECHNICAL FIELD

This invention relates to nanoscale molecule synthesis, including three-dimensional addressable arrays of biopolymeric nucleic acids and processes for manufacturing such arrays. Such arrays can be functionalized with complementary chemical reactive probes to provide catalytic enzymes.

### BACKGROUND ART

This application is related to the following applications, each of which is incorporated herein by reference: U.S. provisional 60/918,144, filed Mar. 15, 2007; U.S. provisional 60/969,154, filed Aug. 30, 2007; U.S. provisional 60/985,961 filed Nov. 6, 2007; U.S. application Ser. No. 11/936,045, filed Nov. 6, 2007; U.S. provisional 61/032,118, filed Feb. 28, 2008; PCT application PCT/US08/57013, filed Mar. 14, 2008; U.S. provisional 61/043,981, filed Apr. 10, 2008; U.S. provisional 61/047,201, filed Apr. 23, 2008; U.S. provisional 61/048,599, filed Apr. 29, 2008; U.S. provisional 61/061,555, filed Jun. 13, 2008, U.S. provisional 61/086,633, filed Aug. 6, 2008; U.S. provisional 61/237,353, filed Aug. 27, 2009.

Scientists and engineers from a wide variety of industries have recently focused on generating nanometer or sub-nanometer construction processes to create diverse capabilities. For example, materials scientists desire to make new materials that exhibit specific properties that can only be created if construction can be controlled at the nanoscale. Chemists and pharmaceutical researchers seek new reactions that depend critically on controlling interactions among molecules that are defined over vectors involving even fractions of angstroms. The difference between success and failure for effective pharmaceuticals can often be measured by these same metrics. Semiconductor engineers seek more compact designs with greater control over electro-magnetic dissonance. In the biofuels industry, nanoscale manufacturing of catalysts can enable the creation of entirely new classes of processes to convert diverse biomass sources into fuel. Each of these applications, along with a great number of others, depends upon a singular required function: the ability to control the placement of diverse molecular functionality in three-dimensional arrays with precise positioning at a sub-nanometer, or even sub-Angstrom scale.

### DISCLOSURE OF INVENTION

The present invention provides a method for enabling the connection of additional elements or molecules to a polynucleobase biopolymer weave comprising incorporating additional chemical linkages within the polynucleobase biopolymer weave. The present invention further comprises such a method wherein at least one of the additional chemical linkages comprises one or more of Amide coupling, ester coupling, or disulfide coupling. The present invention further comprises such a method wherein the polynucleobase biopolymer weave comprises a nucleic acid. The present invention further comprises such a method wherein the nucleic acid comprises at least one of PNA (peptide nucleic acid), DNA (deoxyribonucleic acid), RNA (ribonucleic acid), LNA (locked nucleic acid), GNA (glycol nucleic acid), or TNA (threose nucleic acid).

The present invention further provides a method of generating polynucleobase derivatives, comprising providing an additional chemical linkage aside from the Watson-Crick base pairing link or the backbone link. The present invention further provides such methods, wherein the polynucleobase comprises at least one of thymine, uracil, cytosine, guanine, or adenine.

The present invention further provides methods of generating an arrangement of molecules placed in relation to each other with tolerances of less than about 10 nanometers using any of the methods mentioned herein. The present invention further provides a method of generating a catalyst, comprising using any of the methods mentioned herein to generate a catalyst mimicking known enzymatic transformations but having superior properties to the analogous natural system regarding stability toward pH, temperature, the presence of additives known to accelerate catalysis, or a combination thereof. The present invention further provides a method of generating electrical circuits such as in microprocessors, comprising using any of the methods mentioned herein to produce 3D addressable assemblies of polynucleobases with feature size less than about 1/16 of the feature size currently amenable to scaled fabrication by photolithographic techniques.

The present invention relates to synthesis of a target molecule of the form “Nucleic acid-linker-catalytic residue,” wherein nucleic acid includes cytidine, uridine, adenosine, guanosine and thymidine, as well as any oxidized, reduced or alkylated derivatives of such; linker includes any defined-length organic tethering construct containing nitrogen, oxygen, sulfur, carbon or phosphorus; catalytic residue includes any organic or organometallic moiety capable of catalysis, whether through an acidic, basic, nucleophilic, electrophilic or radical mechanism.

The present invention provides methods to connect the “nucleic acid-linker-catalytic residue” through several types of common methods, including, but not limited to, amide coupling, cycloaddition, ester coupling, and disulfide linking. The primary linkages will be amide linkages, with variations in orientation of amides, and flexibility in terms of amide orientation.

The present invention makes use of methods that are known in the art of peptide chemistry for the creation of amide bonds, namely choice of activated coupling reagent, containing in its structure a carbodiimide, benzotriazol, hydroxysuccinimide group or any related carboxylic acid activator; choice of solvent, most especially DMF, DCM, water, THF; and choice of organic amine base, such as triethylamine or diisopropylethylamine. This invention also makes use of lesser known coupling methods, involving azides, acid chlorides, or anhydrides.

The present invention further concerns a technology to generate what the inventors call “bionanolattices” that allow the construction of stable three-dimensional nanoscale manufacturing platforms. A bionanolattice allows the controllable and predictable placement of diverse molecular functionality, at margins of error measured on the atomic scale. Such a structure can serve as a new nanoscale manufacturing method that can enable the creation of a wide variety of new products in a myriad of industries. As an example, a bionanolattice can be used to specifically place amino-acid side chain functionality in a proper three-dimensional orientation to enable catalysis, mimicking the function of enzymes. These synthetic enzymes, built on the bionanolattice, can exhibit greater activity, resist industrial stress better than native enzymes, and can be applied to the conversion of a wide variety of biomass types (such as bio-waste) to generate fuel. The present invention contemplates a wide variety of applications ranging from MEMS/NEMS construction to diagnostic capabilities to vaccine delivery to materials construction, among others, as potential uses of a bionanolattice.

The present invention further provides methods and apparatuses related to stable three-dimensional nanoscale platforms, including such platforms, methods for making such platforms, and methods of using such platforms. Any of the foregoing may be referred to herein as “DNA Interweave Dimensional Arrays” of “DIDA”, both of which are trademarks of Incitor LLC. As DIDA allows the placement of diverse molecules, compounds, and/or materials on these three-dimensional platforms to tolerances less than a nanometer, a DIDA platform can be functionalized to perform a wide variety of tasks. For example, integrating key amino-acid residues into the DIDA, placing the residues within the appropriate three-dimensional orientation to each other, allows the DIDA platform to exhibit catalytic activity similar to an enzyme. DIDA also enables the construction of complex chemical compounds, where the DIDA platform can “hold” the relevant compounds in place to improve output yield. Other DIDA applications include MEMS/NEMS construction, diagnostic capabilities, vaccine delivery, and others.

The present invention further provides a low cost nanoscale production method that can be applied to a number of industries including energy, semiconductors, pharmaceuticals, chemicals, food and medicine. As an example, the present invention can provide enzymes useful in the conversion of cellulose found in almost any living plant into biofuels. The present invention can use synthetic DNA helices stiffened into metalized three-dimensional weaves to develop and manufacture a wide range of nanoscale constructs, including therapeutic molecules, industrial chemical catalysts and protein vaccines.

### BRIEF DESCRIPTION OF DRAWINGS

The accompanying drawings, which are incorporated in and form part of the specification, illustrate the present invention and, together with the description, describe the invention. In the drawings, like elements are referred to by like numbers.

FIG. 1 is a schematic depiction of synthesis of a target molecule through peptide synthesis.

FIG. 2 is a schematic depiction of synthesis of a target molecule through peptide synthesis.

FIG. 3 is a schematic depiction of a three-dimensional cube made up of DNA wherein three different single strands combine at each corner of the cube (sometimes called a “Chun/Seeman DNA Cube”).

FIG. 4 is a schematic depiction of the concept of a scaffold strand formed into a desired orientation by the incorporation of staple strands.

FIGS. 5A (isometric view) and 5B (front and side views) are schematic illustrations of an example structure, first developed by the inventors, that can be used as a basis for enzyme-like catalysts.

FIG. 6 is a schematic depiction of an example thymidine analogue monomer, with three different side chain formats.

FIG. 7 is a generalized depiction of an example of a structure to be used as a basis for enzyme-like catalysts.

FIG. 8 is a schematic depiction of a sample serine analogue PNA precursor.

FIG. 9 is a schematic depiction of a final example PNA monomer.

FIG. 10 is a schematic depiction of differences and similarities between DNA and PNA.

FIG. 11 is an atomic force microscope image of a DNA weave.

FIG. 12 is a schematic depiction of a folded DNA/PNA weave.

FIG. 13 is a schematic depiction of metallization of DNA/PNA weaves.

FIG. 14 is a schematic illustration of an example ethanol production process according to an embodiment of the present invention.

FIG. 15 is a schematic illustration of cellulosic ethanol production with improved enzymes, according to an embodiment of the present invention.

FIG. 16 is a schematic illustration of corn ethanol production with Improved Enzymes, according to an embodiment of the present invention.

### MODES FOR CARRYING OUT THE INVENTION AND INDUSTRIAL APPLICABILITY

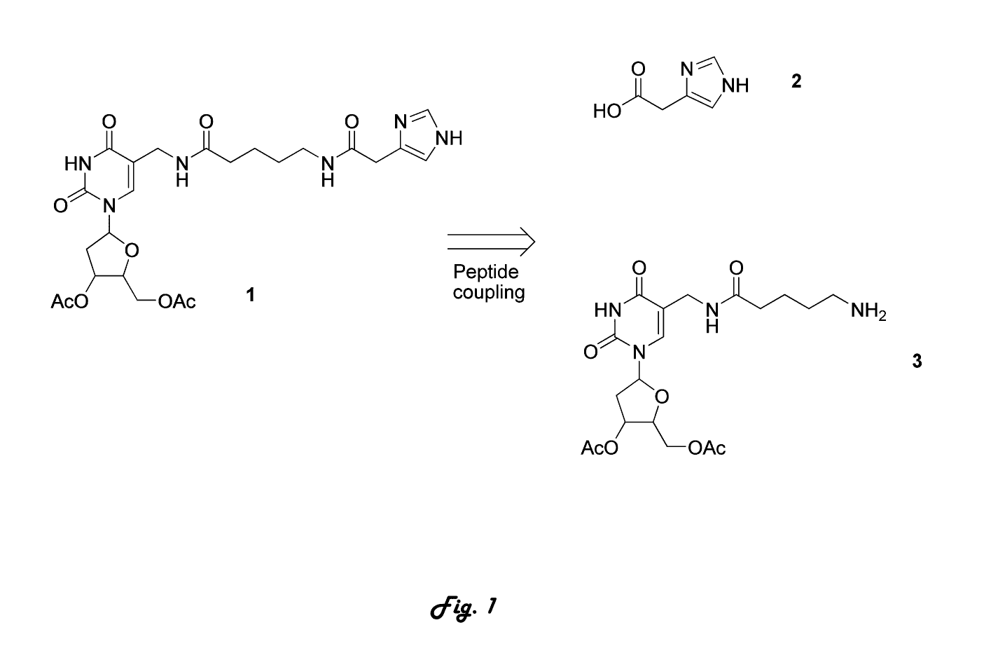

FIG. 1 is a schematic depiction of synthesis of a target molecule through peptide synthesis. In Scheme 1 as depicted in FIG. 1, target compound 1 can arise from 2 and 3, through standard peptide coupling conditions (HOBt, EDCI, DIPEA). Experiments in Scheme 1 have revealed some difficulties, though their resolution could be accomplished through careful chromatographic techniques and reaction conditions. Alternative protecting group manipulations have been shown to alleviate these problems. Alternative approaches are described in the text and drawings below.

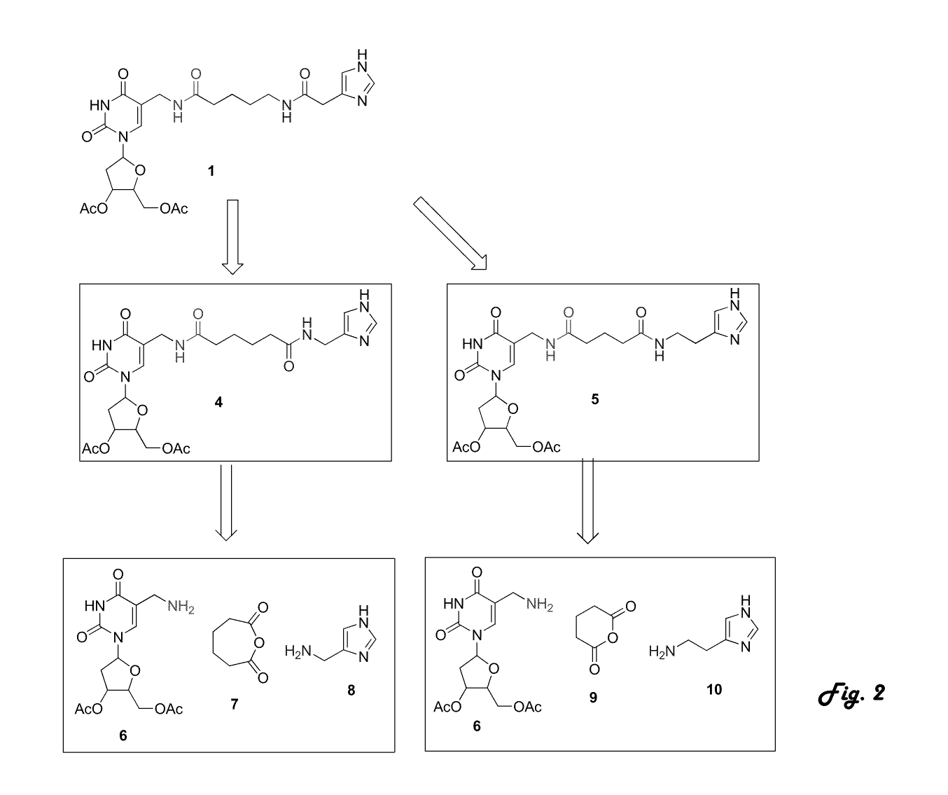

FIG. 2 is a schematic depiction of the synthesis of a target molecule through peptide synthesis. By inverting the blue amide, as seen in 4, the same distance between the imidazole and the thymidine as seen in 1 can be retained. Isomeric compound 5 is also a possibility, and has the advantage of having the natural product histamine 10 as a retron in its synthesis, which is substantially lower in cost than imidazole acetic acid 2. 4 and 5 take advantage of the same common intermediate amine which is currently being used to make 1, however now this can be reacted with anhydrides 7 or 9. This can generate the respective carboxylic acids (not shown), which can then be reacted with 5-(methylamino)-imidazole 8 or histamine 10. This approach can also eliminate the need for protecting groups.

Bionanolattices. Building a bionanolattice can be described as five steps:

Producing three-dimensional structures made up of cross-linked strands of a biopolymer (herein referred to as the “bionanolattice”), such as deoxyribonucleic acid (DNA);

Producing hybrid monomers of a biopolymer such as DNA or peptide nucleic acid (PNA) that contain a covalently bonded side chain displaying a chemically linkable group (herein referred to as the “monomer stud”);

Connecting a chemical linker, either synthesized or commercially obtained, to the side chain of the monomer stud that enables spacing, orientation, electrostatic properties and/or a range of steric motion control at the nanoscale (herein referred to as the “linker”);

Covalent attaching of a catalytic functionality of interest to each linker, which will serve as an acid, base, electrophile, nucleophile or single electron acceptor or donor. Non-catalytic functionality may also be utilized to bolster or disfavor hydrogen-bonding, π-stacking, or other non-bonding interactions.

Incorporating each monomer stud, complete with linker and catalytic molecule of interest connected, within the bionanolattice (herein referred to as the “loaded bionanolattice”).

For the purposes of the present description, the sample application of constructing catalysts that exhibit function analogous to enzymes will be used. In addition, the term “molecule” will be used to reference a molecule, compound, or other material in a general sense. This enzyme application is merely an example, however, of the wider applicability of a bionanolattice.

Production of a Bionanolattice. The construction of a bionanolattice is based upon a native characteristic of certain biopolymers such as DNA wherein the biopolymer forms a double helix (or other multiple helix) based upon lowest-energy thermodynamic considerations set forth by Watson-Crick base pairing of nucleobases within each helix. This base pairing characteristic can be exploited to generate junctions and cross-links between different helical domains (meaning a single double helix or multiple helix strand) to form complex geometrical designs. A number of junction types have been discovered, such as the Holliday Junction (wherein four single strand DNA sequences form a square crosslink), that enable different numbers of single strands of DNA to combine in predictable manners, forming the building blocks for three-dimensional structure design (commonly referred to as “weaving”). FIG. 3, as an example, shows a three-dimensional cube made up of DNA wherein three different single strands combine at each corner of the to form a construct referred to as the “Chen/Seeman DNA Cube” (J. Chen and N. C. Seeman, The Synthesis from DNA of a Molecule with the Connectivity of a Cube, Nature 350, 631-633 (1991), incorporated herein by reference).

A modification of the biopolymer weave concept, called DNA Origami, was generated by Dr. Paul Rothemund of the California Institute of Technology (P. W. K. Rothemund, Folding DNA to create nanoscale shapes and patterns, Nature, 440, 297-302 (2006), incorporated herein by reference). DNA Origami follows the basic concepts of biopolymer weaving, but incorporates the concept that one of the two helices within the double helix is a single long single strand of DNA (called a “scaffold strand”) that is “folded” into diverse shapes based upon corresponding synthetically derived “staple strands.” FIG. 4 demonstrates the concept of a scaffold strand formed into a desired orientation by the incorporation of staple strands.

The black strand in FIG. 4 is a single long scaffold strand consisting of a sequence of nucleotides that does not repeat over a seven or eight base pair sequence. The blue and red strands are staple strands. Due to the unique sequence of base pairs, the staple strands enforce the scaffold strand in an adopted desired shape. This concept, extended into three-dimensions, allows for the cost-effective construction of complex three-dimensional structures. However, as three-dimensional biopolymer weaves alone have little utility, they have not previously been successfully applied to commercial applications at a production scale.

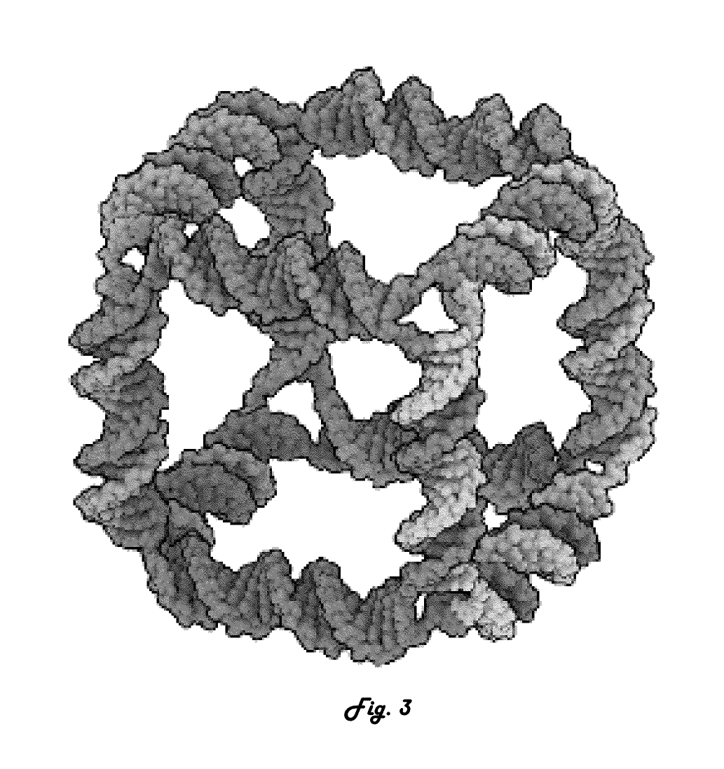

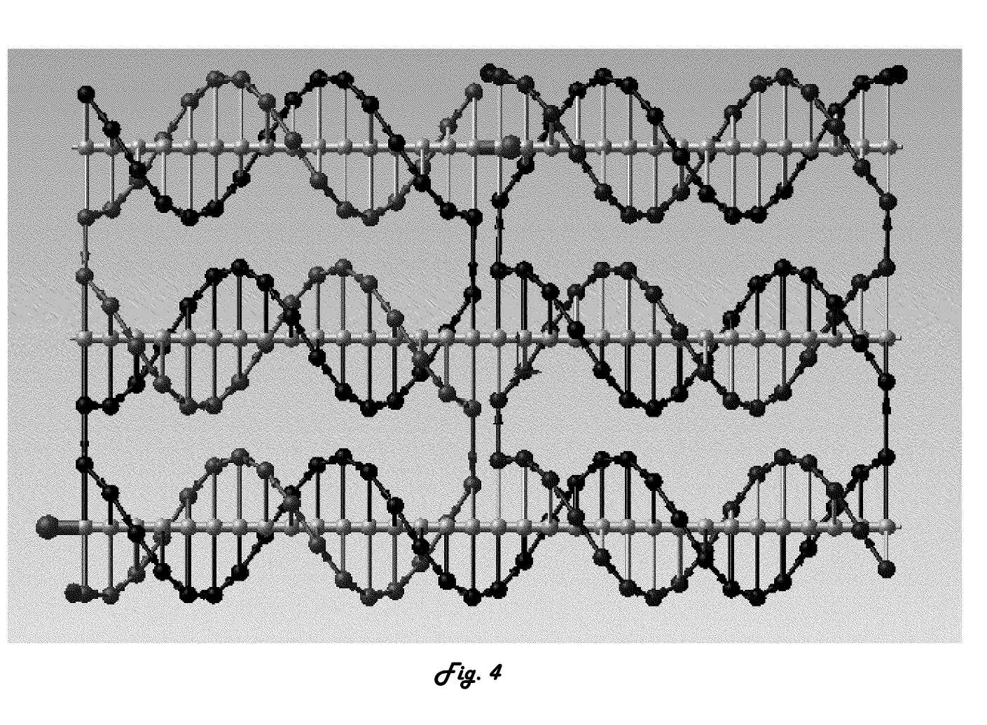

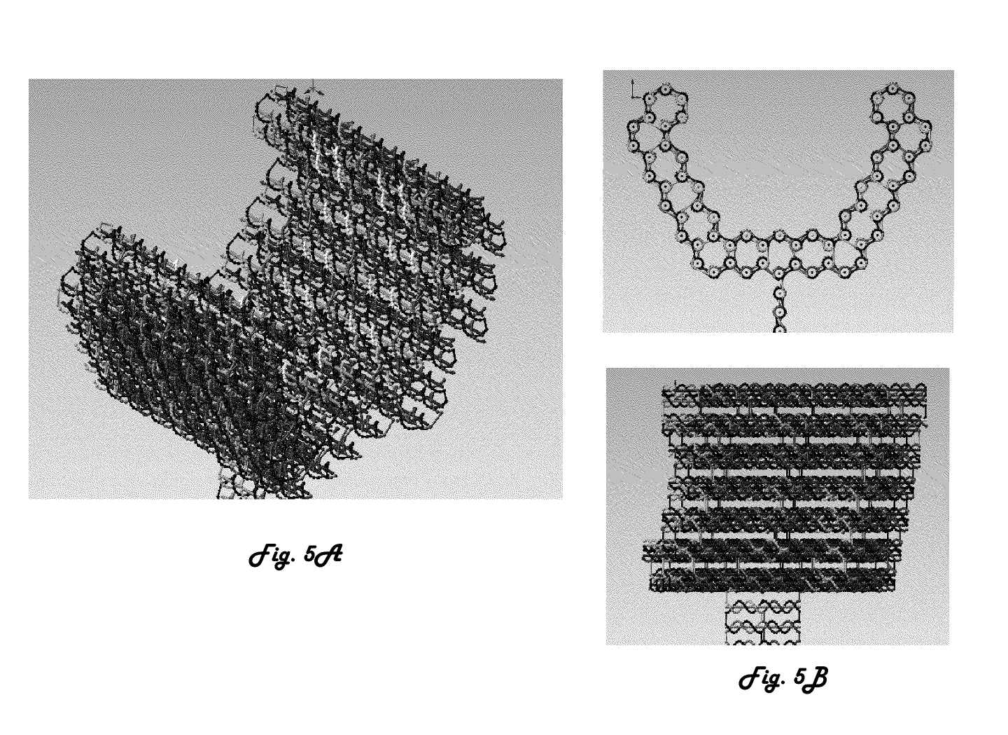

The present invention builds on the concept of three-dimensional DNA weaves and DNA Origami and functionalizes them, adding in the ability to connect diverse molecules of interest to various points in the structure. One example of such a structure, first developed by the inventors, to be used as a basis for enzyme-like catalysts is shown in FIGS. 5A and 5B.

The DNA design illustrated in FIGS. 5A and 5B comprises a structural strand of DNA derived from the M13mp18 viral genome from the single-stranded, male-specific filamentous DNA bacteriophage M13, with a number of synthesized 200-mer staple strands that bind the structural strand into the desired configuration. The design is optimized so that when the structural strand and staple strands are placed in solution, the structure is adopted as a consequence of the equilibrium thermodynamics. The structure example from FIGS. 5A and 5B incorporates two important characteristics: rigidity and predictability. The double-wall design ensures that the shape remains static within a wide range of ambient conditions. As the DNA sequences are known, the structure is predictable—any sequence location within the three-dimensional structure is known to within a nanometer on all three axes. The structure is less than 50 nanometers wide and 30 nm deep in the front view and 40 nm long in the side view.

By studding the design's interior with linkers and biomimetic functionality, the present invention anticipates being able to mimic the catalytic performance of an enzyme active site. By generating additional bionanolattice platform designs, or changing the molecule type or location on the sample design, different functions can be achieved, including, but not limited to, a wide variety of enzyme mimics, diagnostic solutions, pharmaceuticals, electromagnetic materials, or other novel applications. The concept does not require DNA as the biopolymer, but as DNA is currently relatively inexpensive, is used as an example of the general potential.

Production of Monomer Studs. The bionanolattice platform, based upon woven biopolymers, provides the predictability and stability required to enable nano-precise three-dimensional placement of desired molecules. Given the predictable nature of the structure, the precise placement of amino acid side-chain functionality can be greatly assisted by computational design and molecular modeling. An additional capability can be required, however. To connect molecules of interest to the bionanolattice and thereby achieve a desired function, either the original biopolymer structure can be constructed out of monomers that contain side chains to provide a location to connect functionality of interest, or parts of the biopolymer structure can be replaced with monomers that contain side chain functional groups. Using a PNA analogue that contains the necessary side chain is an option. However, synthesizing chains of PNA is generally more costly than alternatives such as DNA.

An alternative is to construct the bionanolattice out of a biopolymer such as DNA that is less costly to synthesize, then replace some of the DNA with shorter strands of PNA. There is still a cost concern, albeit a smaller one, based upon the PNA synthesis requirements. In addition, the helical structure of PNA natively forms at fifteen (15) base pairs per turn, whereas DNA natively forms at 10.5 base pairs per turn. This difference stresses the overall bionanolattice structure, potentially reducing its integrity. The inventors have explored generating a PNA monomer based upon serine to provide a side chain, and the inventors have generated a DNA nucleotide derivative, adding the required side chain to thymidine. This can resolve the base pair per turn issue, and potentially dramatically lower the cost of an activated bionanolattice.

The approach replaces selected thymidine nucleotides within the staple strands with a designed, synthetic thymidine derivative. This can allow any location of a thymidine to serve as a stud monomer, wherein a linker and a molecule of interest could be connected. In the case of the synthetic enzyme example, the molecule of interest can be an amino-acid or a metallic ion.

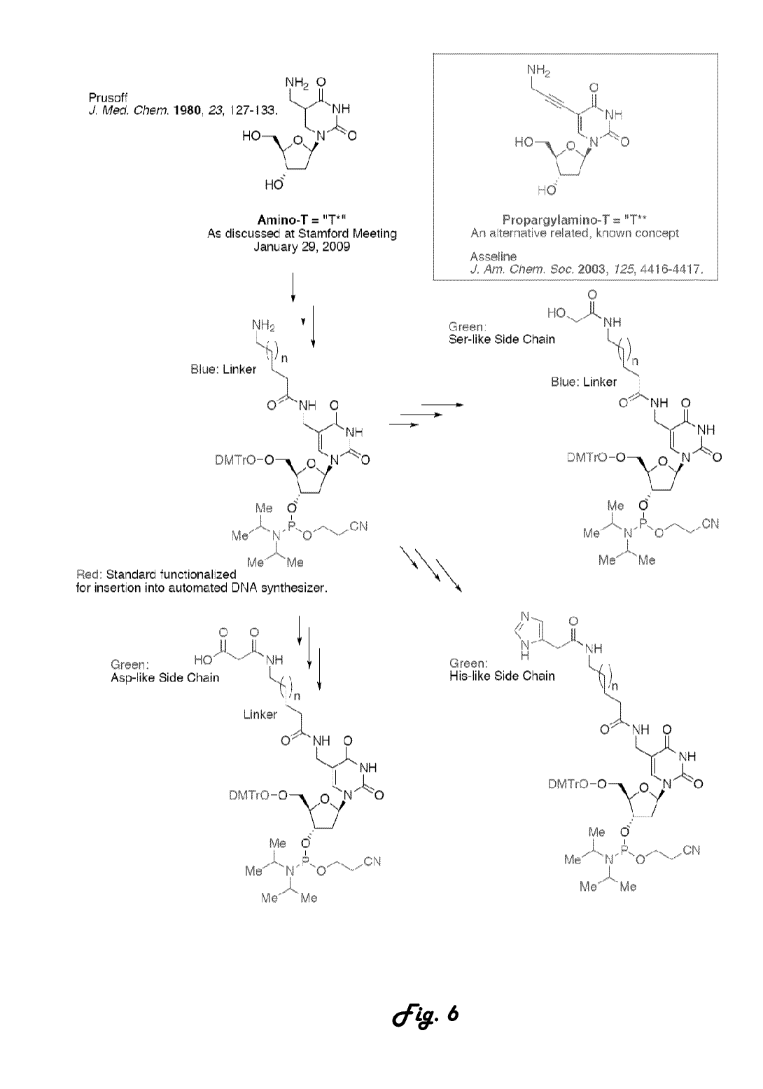

FIG. 6 is a schematic depiction of an example thymidine analogue monomer, with three different side chain formats. The black segments of each molecule show the core thymidine elements. The blue segments could vary due to the type of linker required. The green components show the new side chain functionalities, now constitutive of the thymidine analog, which can then be connected to the desired molecular component containing desired catalytic functionality. As the location of each thymidine within the bionanolattice is known, by varying the parameters of the linkers (length, flexibility, etc), a desired functional group can be placed in a specific location in three-dimensional space, relative to the structure of the bionanolattice. The functional groups of interest can be added to the side chain in advance of insertion into the bionanolattice, or after. If before, the stud monomer, with molecule and linker attached, can be synthesized in a single strand of DNA to provide selectivity in placement within the bionanolattice. Alternatively, different protection groups can be applied to the stud monomer (the thymidine) side chain to enable deprotection and synthesis of the molecule to the side chain after insertion into the bionanolattice. In either case, the technique provides:

1) A location within the bionanolattice where diverse functionalities can be attached;

2) Predictability of the location;

3) Selectivity of the location.

Either the PNA or the thymidine analog approach can accomplish this end, along with other biopolymers that have monomer side chains or can be derivitized to have monomer side chains. If using DNA, the approach of adding a side chain is also not limited to thymidine, but can be applied to adenosine (or deoxyadenosine), guanosine (or deoxyguanosine), cytidine (or deoxycytidine), or uridine (deoxyuridine) as needed. The thymidine description above is used merely as an example.

The concept, then, in generating locations where the predictable placement of molecules within the bionanolattice can occur can be described as three-fold:

1) Generate modified biopolymer monomers that contain a side chain (the stud monomers);

2) Connect chemical linkers and molecules of interest to the side chain;

3) Replace segments of the staple strand (or structural strand in some cases) of the bionanolattice with the stud monomers.

The stud monomers can be synthesized variations on PNA, DNA or other biopolymers. Selective placement of the stud monomers within the bionanolattice can be accomplished via inserting the stud monomers into the staple strands during synthesis. The uniqueness of the coded sequence of the staple strand will ensure that the stud monomer is predictably placed in the bionanolattice. Details of positioning can be readily assisted using computational molecular design programs. Selectivity of molecule attached to each stud monomer can be achieved by either connecting the molecule to the stud monomer prior to staple strand synthesis, or generating a unique protection type for the stud monomer in question.

The concept of replacing DNA with PNA within DNA cross-linked structures has been explored by Dr. Ned Seeman of NYU with success, highlighting the feasibility of this approach. P. S. Lukeman, A. Mittal, & N. C. Seeman, Two Dimensional PNA/DNA Arrays: Estimating the Helicity of Unusual Nucleic Acid Polymers, Chemical Communications, 1694-1695 (2004), incorporated herein by reference. A number of groups have generated derived versions of nucleotides. U. Asseline, V. Roag, Oligo-2′-deoxyribonucleotides containing uracil modified at the 5-position with linkers ending with guanidinium groups, Journal of the American Chemical Society, 125, 4416-4417 (2003), incorporated herein by reference.

Combinatorial Chemistry Development. Aside from the commercial production benefits deriving from structures more resistant to ambient stresses such as extremes in salinity, pH, and/or temperature, a specific benefit related toward continuous improvement of desired functions emerges. An activated bionanolattice, by allowing specific control over the placement of each molecule within three dimensional space, allows researchers to more rapidly generate new functions. Using the synthetic enzyme example, a researcher can easily vary the 3D orientation, selection, or other characteristics of amino-acids within the bionanolattice, on a directed or randomized basis to generate new enzymes that may exhibit improved catalysis profiles. In addition, as the bionanolattice is not limited to the naturally occurring amino-acids as required in micro-organism produced options, new synthetic enzymes, metallic ions, or other catalysis enhancers can for the first time be incorporated into a commercially viable enzyme.

Three-dimensional Nanoscale Platforms. The present invention further provides methods and apparatuses related to stable three-dimensional nanoscale platforms, including such platforms, methods for making such platforms, and methods of using such platforms. Any of the foregoing may be referred to herein as “DNA Interweave Dimensional Arrays” of “DIDA”, both of which are trademarks of Incitor LLC. As DIDA allows the placement of diverse molecules, compounds, and/or materials on these three-dimensional platforms to tolerances less than a nanometer, a DIDA platform can be functionalized to perform a wide variety of tasks. For example, integrating key amino-acid residues into the DIDA, placing the residues within the appropriate three-dimensional orientation to each other, allows the DIDA platform to exhibit catalytic activity similar to an enzyme. DIDA also enables the construction of complex chemical compounds, where the DIDA platform can “hold” the relevant compounds in place to improve output yield. Other DIDA applications include MEMS/NEMS construction, diagnostic capabilities, vaccine delivery, and others.

Building DIDA arrays comprises three steps:

Producing three-dimensional structures made up of cross-linked DNA strands;

Producing peptide nucleic acid (PNA) monomers that connect to chemical linkers that in turn connect to various molecules, compounds or materials of interest;

Incorporating the PNA strand into the 3D woven DNA structure.

For the purposes of this description, the sample application of constructing catalysts that exhibit similar functionality as that of enzymes will be used. In addition, the term “molecule” will be used to reference a molecule, compound, or other material in a general sense. This enzyme application is merely an example, however, of the wider applicability of DIDA, and the invention(s) described herein include the methods described, the platforms described, the use of such platforms, and all subsets, variations, and extensions to any of the foregoing that one skilled in the art will appreciate from reading the present disclosure.

Production of 3D DNA Structures. DIDA technology uses a native characteristic of DNA wherein certain DNA sequences naturally exhibit junctions and cross-links to form complex geometrical designs. A number of junction types have been discovered, such as the Holliday Junction (wherein four single strand DNA sequences form a square crosslink) that enable different numbers of single strands of DNA to combine in predictable manners, forming the building blocks for three-dimensional structure design. FIG. 3, for instance, shows a three-dimensional cube made up of DNA wherein three different single strands combine at each corner of the cube.

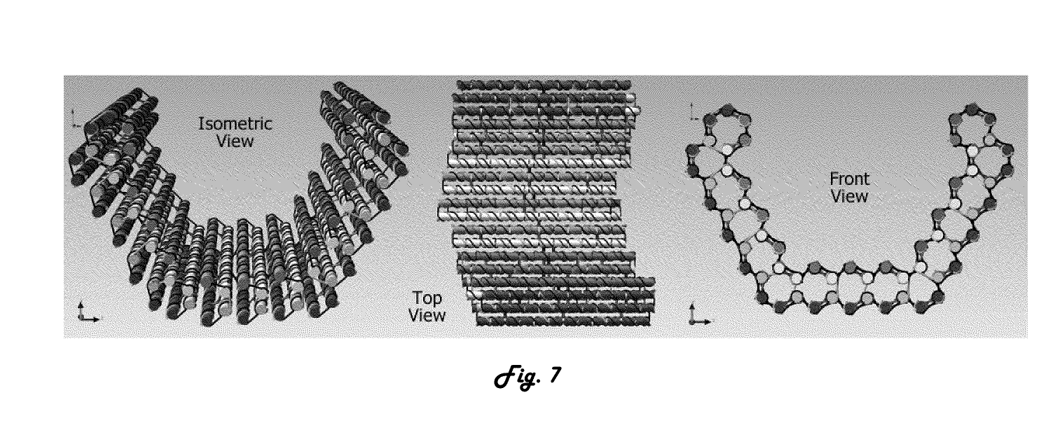

Although the technology for generating three-dimensional structures from DNA has been in existence for a period of time, it has not been successfully applied to commercial applications to date. DIDA uses general concepts of three-dimensional DNA weaves, or DNA origami, and applies them to the creation of structures that can serve as scaffolds for diverse functionality. One example of such a structure to be used as a basis for enzyme-like catalysts is shown in a generalized view in FIG. 7.

The DNA design in FIG. 7 comprises a structural strand of DNA derived from the M13mp18 viral genome from the single-stranded, male-specific filamentous DNA bacteriophage M13, with a number of synthesized 200 mer “staple strands” that bind the structural strand into the desired configuration. The design is such that when the structural strand and staple stands are placed in solution, the structure naturally forms. The structure example from FIG. 2 incorporates two important characteristics: rigidity and predictability. The double-wall design ensures that the shape remains static within a wide range of ambient conditions. Since the DNA sequences are known, the structure is predictable—any sequence location within the three-dimensional structure is known to within a nanometer on all three axes. The structure is less than 50 nanometers wide and 30 nm deep in the front view and 40 nm long in the top view.

By “studding” the design's interior with amino-acids and chemical linkers, the structure can be used to mimic the functionality of an enzyme active site. Additional DIDA platform designs, or changing the “studded” molecule type or location on the sample design, can provide different functions, including a wide variety of enzyme mimics, diagnostic solutions, pharmaceuticals, or other novel applications.

Production of PNA Monomers. The use of DNA within a DIDA platform provides the predictability and stability desired to enable nano-precise three-dimensional placement of desired molecules. Actually connecting these molecules of interest, however, requires a modification of some of the DNA sequence. Examples include generating custom DNA monomers with modified backbones to provide a “free” connection open for chemical reactions, or introducing Peptide Nucleic Acid custom monomers in place of certain DNA sequences, among others.

PNAs mimic the function of DNA, yet have greater stability over a wider range of pH and are a more sequence specific binder. Utility has been found in the modulation of genes pertinent to disease, with glycine based PNA in common use. In the case of catalyst design as in the example in this document, while chemical stability is desired, the portion of the PNA derived from the amino acid side chain adopts a critical role in the key properties of the molecule.

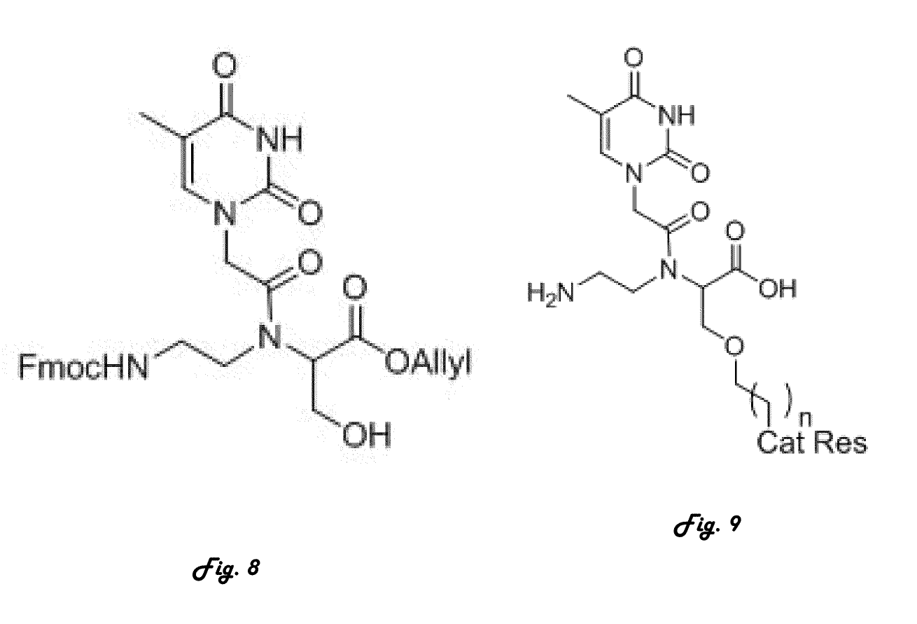

DIDA can be used to construct a custom serine analogue PNA precursor with two protected side chains that contains an additional hydroxyl group (hence the use of serine as an analogue). The additional hydroxyl group would allow for molecules to be connected to the monomer, where the monomer is placed within a specific location within the DIDA platform using the “standard” side chains. A sample monomer precursor is shown in FIG. 8.

Creating a custom PNA monomer with three side chains provides predictable locations within the DIDA platform that can connect to molecules not necessarily related to the DNA/PNA weave itself. For instance, the unprotected hydroxyl side chain can be bound to a chemical linker of varying length, angle, flexibility and other characteristics. These chemical linkers can in turn be bound to molecules of interest. The linkers can provide spacing and orientation to ensure that the molecules of interest react in the desired manner and maintain a spatial orientation to each other as needed. In the example of an enzyme mimic, the molecules can be amino-acids that are equivalent to the amino-acids within a given enzyme's active site. The linkers can be steroidal to maintain the appropriate three-dimensional orientation of the amino-acids to each other, resolving the “folding” challenge inherent in alternative synthetic enzyme design. Improved enzymes can be generated by changing the linkers, the amino-acids connected to the linkers, or the location of the hydroxyl groups on the core platform.

In the continued example of synthetic enzyme construction, once the linker and catalytic residue are connected to the free hydroxyl, the precursor can be unprotected to form a final PNA monomer, as shown in FIG. 9. These monomers can be synthesized in single strands of PNA, some monomers of which can be more traditional glycine analogue PNA, and some of which can be the above-described serine analogue PNA with the molecules of interest pre-attached.

DIDA accordingly allows generation of locations where the predictable placement of molecules within the DIDA structure can occur with three steps:

1) Generate modified nucleic acid monomers that contain an additional side chain;

2) Connect chemical linkers and molecules of interest to the side chain;

3) Replace segments of the staple strand (or structural strand in some cases) of the DIDA structure with single strand PNA that contains modified monomers with the molecules of interest attached. The location of the PNA monomers within the DIDA platform, with the characteristics of the chemical linker, will define the placement of the molecules of interest within three-dimensional space.

The concept of replacing DNA with PNA within DNA cross-linked structures has been explored by Dr. Ned Seeman of NYU with success.

Incorporation of PNA into the DIDA Platform. The initial DIDA platform DNA design incorporates locations that have been planned to be replaced with PNA strands of 18-20 mer in length. To complete an entire functionalized DIDA structure, once the PNA monomers have been attached to the molecules of interest (in the example of synthetic enzymes, these molecules can be amino-acids), a strand of PNA consisting of the monomers with glycine or other analogue PNA monomers can be synthesized using industry standard practices. These PNA strands can be placed in solution with the DNA staple and structural strands. The nature of the design is that the strands will natively shape themselves into the desired configuration. A functionalized DIDA structure has been built.

Combinatorial Chemistry Development. Aside from the commercial production benefits deriving from structures more resistant to ambient stresses such as extremes in salinity, pH, and/or temperature, a specific benefit related toward continuous improvement of desired functions is enabled by DIDA. DIDA, by allowing specific control over the placement of each molecule within three dimensional space, allows researchers to more rapidly generate new functions. Using the synthetic enzyme example, a researcher can easily vary the 3D orientation, selection, or other characteristics of amino-acids within the DIDA structure, on a directed or randomized basis to generate new enzymes that may exhibit improved catalysis profiles. In addition, as DIDA is not limited to the naturally occurring amino-acids as required in micro-organism produced options, new synthetic enzymes, metallic ions, or other catalysis enhancers can for the first time be incorporated into a commercially viable enzyme. DIDA provides a method of researching, developing and mass-producing at a nanoscale that can impact a wide variety of industries.

The following references, each of which is incorporated herein by reference, can facilitate understanding of the preceding description: J. Chen and N. C. Seeman, The Synthesis from DNA of a Molecule with the Connectivity of a Cube, Nature 350, 631-633 (1991); Rothemund, P. W. K., Folding DNA to create nanoscale shapes and patterns, Nature, 440, 297-302 (2006); Nielsen, P. E.; Haaima, G., Peptide nucleic acid (PNA). A DNA mimic with a pseudopeptide backbone, Chem. Soc. Rev., 26, 73-78 (1997); Venkatesan, N.; Kim, B. H., Peptide conjugates of oligonucleotides: Synthesis and applications, Chem. Rev., 106, 3712-3761 (2006); Porcheddu, A.; Giacomelli, G., Peptide Nucleic Acids (PNAs), A Chemical Overview, Curr. Med. Chem., 12, 2561-2599 (2005); P. S. Lukeman, A. Mittal, & N. C. Seeman, Two Dimensional PNA/DNA Arrays: Estimating the Helicity of Unusual Nucleic Acid Polymers, Chemical Communications 2004, 1694-1695 (2004). Each of the foregoing references is incorporated herein by reference.

Nanoscale Production with DNA Weaves. The present invention further provides a low cost nanoscale production method that can be applied to a number of industries including energy, semiconductors, pharmaceuticals, chemicals, food and medicine. As an example, the present invention can provide enzymes useful in the conversion of cellulose found in almost any living plant into biofuels. The present invention can use synthetic DNA helices stiffened into metalized three-dimensional weaves to develop and manufacture a wide range of nanoscale constructs, including therapeutic molecules, industrial chemical catalysts and protein vaccines.

In some embodiments of the present invention, amino acids, the building blocks of enzymes, are attached to specific locations within woven DNA structures. These weaves are chemically modified to impart three dimensional form and then are hardened with the application of metal ions that adhere to the DNA. The metallic DNA weaves retain their shape, holding the amino-acids in a specific three dimensional orientation to mimic the catalytic structure of an enzyme. In production, trillions of enzymes can be created simultaneously using this method in hours. Random or directed amino-acid variations can be added to the weave.

Given the benefit potential in both production cost and better activity that building enzymes synthetically can afford, many entities have experimented with different methods for creating synthetic enzymes. No one has been able to create a universal technique for generating synthetic enzymes (although a few specific small enzymes have been created) that exhibit high levels of catalytic activity due to three principal challenges:

Sequence Construction: Enzymes contain up to thousands of amino-acids in a specific sequence. Current synthetic methods (Merryfield Peptide Synthesis) of combining amino-acids cannot generate commercial quantities in sequences exceeding 100 amino-acids;

Folding: To exhibit catalytic activity, amino-acid sequences must be “folded,” or placed into specific three-dimensional orientation. No one previously has been able to control the fold of amino-acid sequences of the necessary length;

Movement: Even if the amino-acid sequences have been folded, the orientation changes over the course of the catalytic reaction. No one previously has been able to study this with synthetic amino-acids as yet due to the inability of others to solve the folding challenge.

The present invention can resolve these three issues by simplifying to the core issue and using nanotechnology physical techniques instead of biochemical techniques. The present invention can eliminate most of the amino-acids in a given enzyme, concentrating only on those amino-acids actively involved in catalysis, eliminating the need to build long sequences. It can control the placement of these amino-acids in three-dimensional space using a hardened weave of DNA and PNA (Peptide Nucleic Acid), to within a nanometer, resolving the folding challenge. The technology enables these specific amino-acids to move within predictable paths, solving the movement requirement.

The present invention can be used to create and produce new enzymes for example for the ethanol market. The present invention can be also used to create many three dimensional structures at nanoscale levels that require precise molecular placement, including therapeutic biological molecules, industrial chemical catalysts and miniscule electronic components, more efficiently than current processes.

The present invention provides the following key benefits to the ethanol market:

It enables the rapid development of hyper-efficient enzymes orders of magnitude more effective than currently available products;

It enables enzymes to be introduced at various stages in the ethanol production process, improving production efficiency and reducing costs.

The present invention takes advantage of several fundamental biological rules:

Enzymes are composed of polymers of amino-acids (polypeptides) and other molecules;

Amino-acids can be coupled to Peptide Nucleic Acids, or PNA;

PNA can connect to DNA;

Specific DNA sequences natively form woven structures at a nanoscale;

To exhibit catalytic capability, enzyme polymers must be “folded” in a specific three-dimensional orientation.

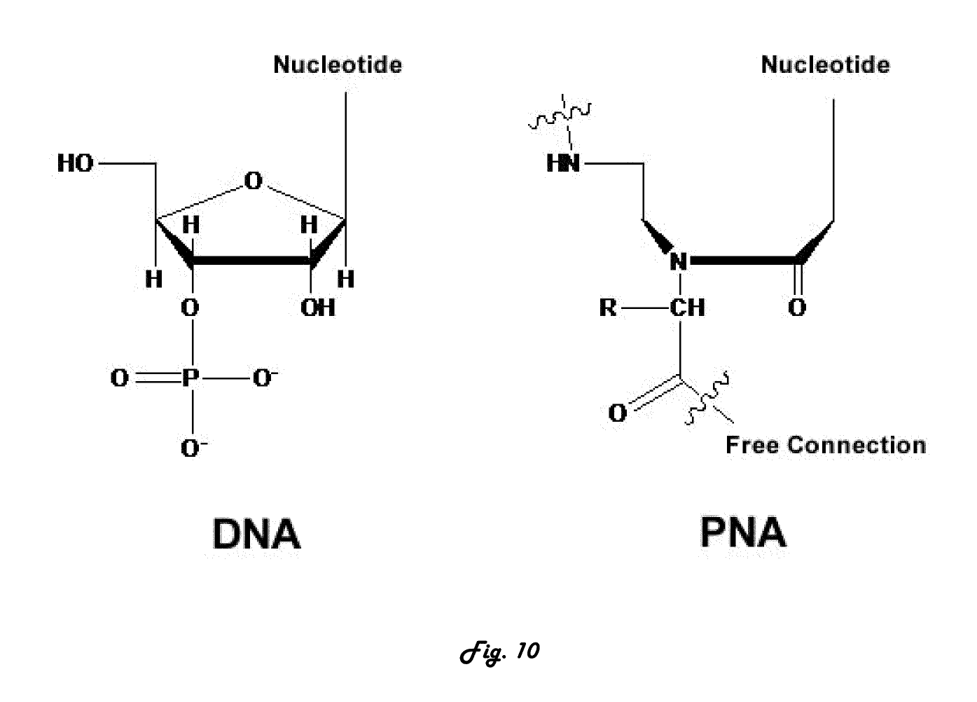

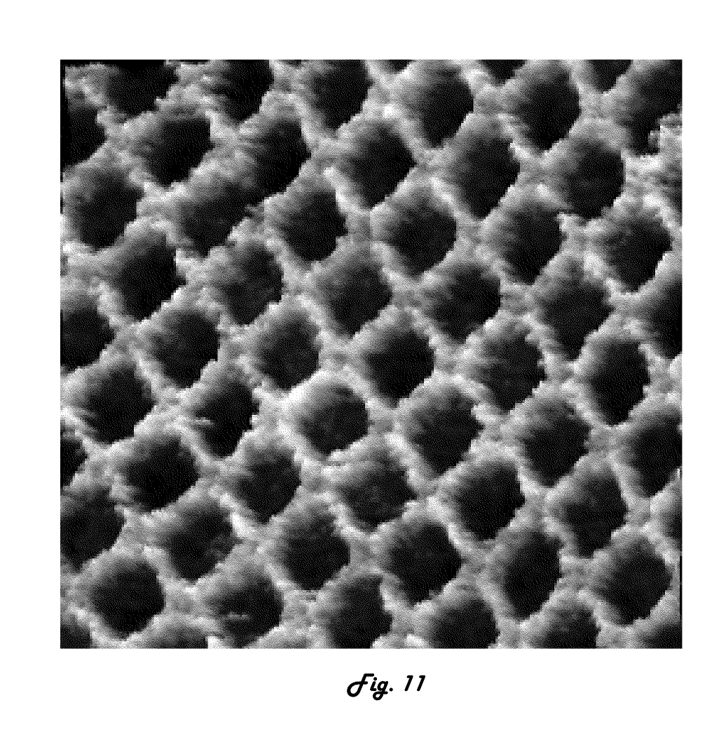

The present invention capitalizes on these biological rules. The process starts with the creation of Peptide Nucleic Acid (PNA) sequences. PNA is related to DNA in that it consists of the same nucleotides as DNA, but has a peptide (amino-acid) backbone instead of the deoxyribose (a sugar) backbone existing within DNA. FIG. 10 demonstrates the differences (and similarities) between DNA and PNA.

Embodiments of the present invention can use PNA since, as can be seen from the figure, molecules can be attached to both sides of PNA. One side contains the nucleotide base, which will attach to DNA or other PNA molecules. The other is able to be chemically bonded to a variety of substances, including natural or synthetic amino-acids, metals, or other organic or inorganic compounds. This unique capability allows the present invention to be used to design PNA sequences that contain catalytically important molecules, and precisely control the placement of these molecules along the sequence of PNA.

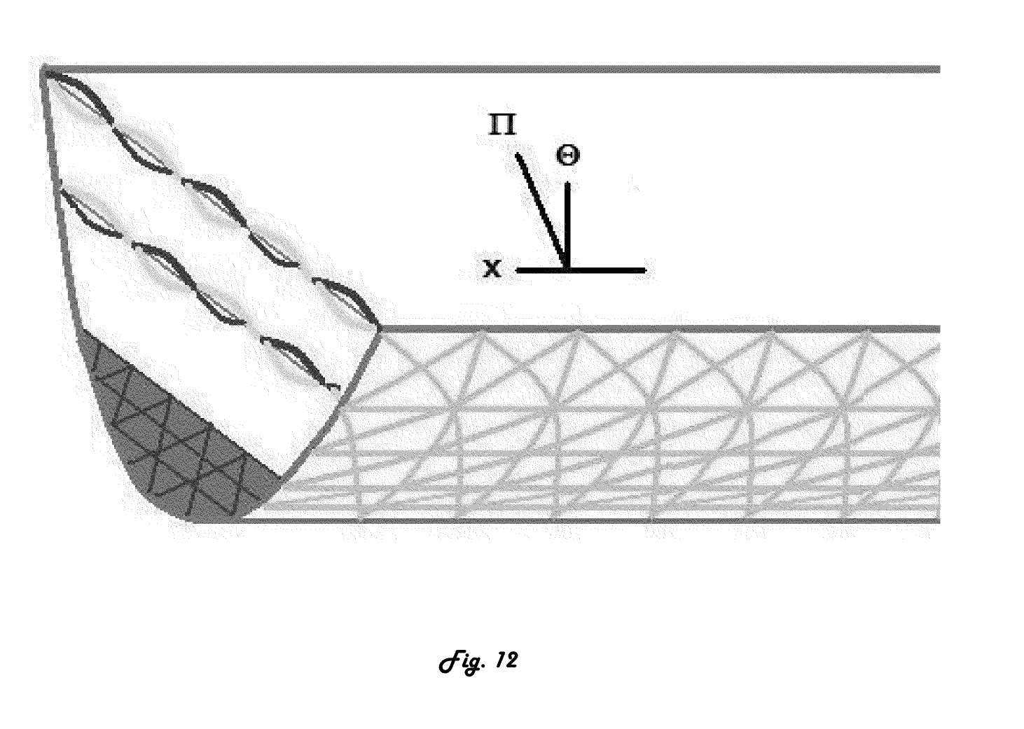

Once the PNAs with catalytic molecules attached are built, a weave of double-stranded DNA can be constructed. The PNA can be embedded within the weave, replacing some of the DNA sequence. By controlling the PNA sequence of nucleotides, the PNA sequence can be precisely placed within two dimensional space within the weave. As the molecule of interest is attached to the PNA in a known configuration, the molecule is also precisely placed within two dimensional space. FIG. 11 shows an atomic force microscope image of a DNA weave. See, e.g., http://www.cs.duke.edu/~reif/paper/SELFASSEMBLE/DNAlattice.Overview.ppt, visited Sep. 13, 2008, incorporated herein by reference.

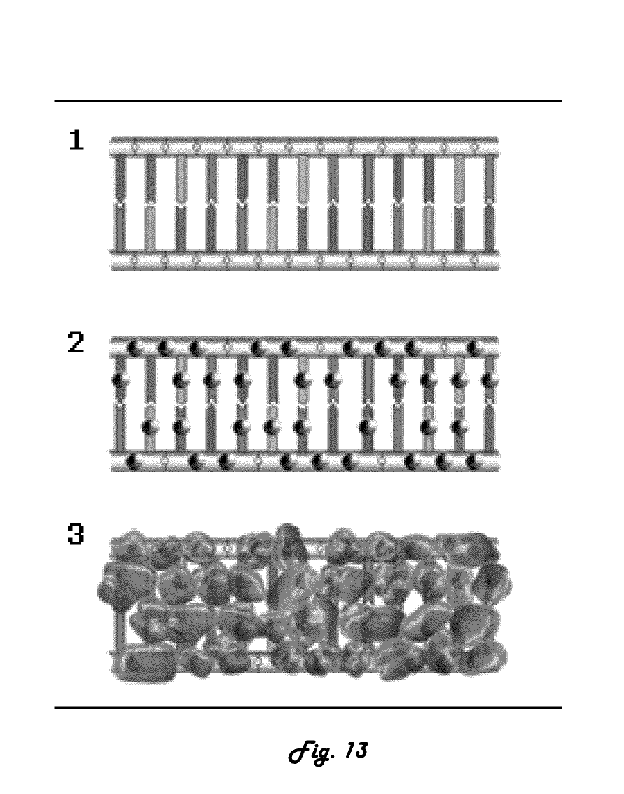

To gain three dimensional placement, PNAs containing compounds that attract to each other can be placed on the outer edges of the weave, “folding” the entire structure into a semi-cylinder. FIG. 12 demonstrates this concept.

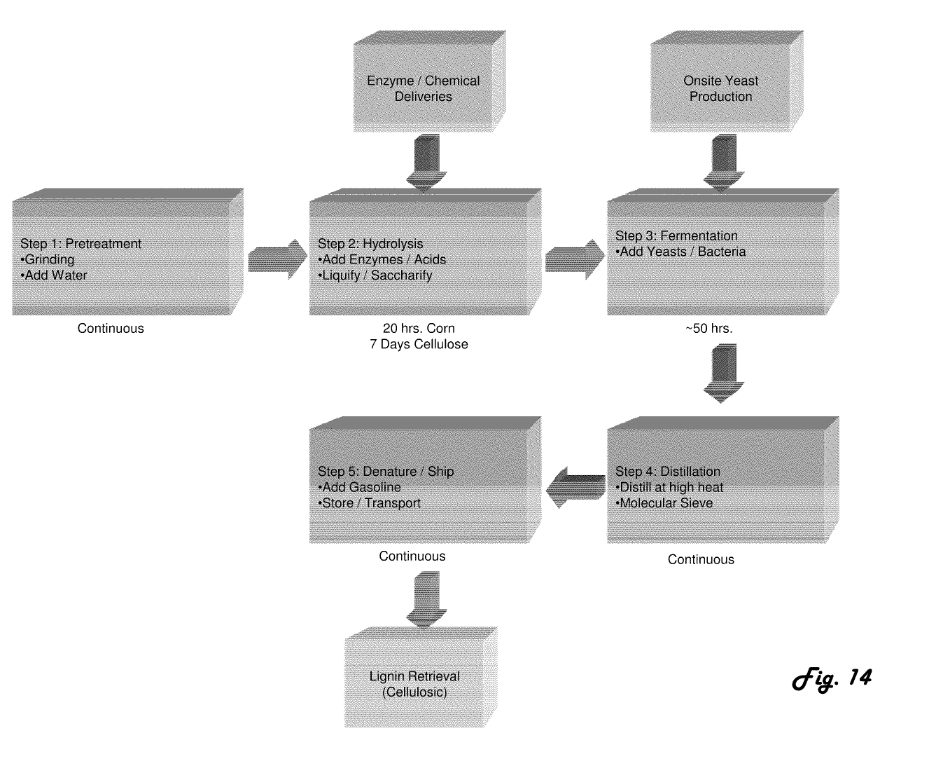

The final production step involves adding metallic ions in solution to the weave, to harden it permanently into the semi-cylindrical shape. As DNA backbones as well as nucleotides are net negatively charged, they attract and bind to positively charged metal ions such as aluminum ions. The metal ions surround the DNA lattice, imparting rigidity. FIG. 13 shows the process. See http://www.integratednano.com/tech-core-htbw.asp#, visited Sep. 13, 2008, incorporated herein by reference.

The top portion of FIG. 13 shows a standard double strand of DNA. The middle portion of FIG. 13 shows metal ions attracted to the negative charge of the different components of the DNA strand. The bottom portion of FIG. 13 shows how additional metal chemically adheres to the initial metal ions, sheathing the original DNA strands in a rigid metallic cover.

Embodiments of a process according to the present invention can be a chemical/physical methodology, as compared with a biological process. A three-dimensional weave as contemplated in the present invention can eliminate the need for the vast majority of amino-acids in a biologically produced enzyme that exist solely to provide a given 3D conformation while providing the appropriate 3D structure. As the amino-acids used in the present invention can be automatically attached to the weave in the correct location with chemical compounds that control the movement capabilities of the amino-acids, structures produced according to the present invention can account for the needs of catalysis. The process can be faster and more cost effective than traditional methods.

In addition to providing a rapid and highly-efficient method to generate enzymes, an abiotic metallic process according to the present invention can inherently produce enzymes that withstand higher temperatures and pressures, as well as wider extremes of pH, salt and shear forces which characterize industrial processes.

The benefits of this method of production are extensive. An enzyme produced according to the present invention:

Can be used in catalysis multiple times without loss of integrity, as the amino-acids are permanently affixed to a physical metallic construct;

Eliminates the need to maintain bacteria or fungi to produce enzymes;

Generates enzymes that are more active in less time;

Generates enzymes that are more resistant to industrial stresses;

Can generate and improve upon ANY known enzyme, not limited to those relevant for fuel production;

Reduces the energy cost required to produce enzymes under current processes.

The ability to control the genetic sequence within the weaves also provides extensive research and development benefits that reach into other processing industries. Aside from producing millions of identical enzymes in a single process, the present invention also enables the production of millions of different enzymes simultaneously. Thus the present invention can be used to generate new and more efficient enzymes. By introducing minor random changes into the amino-acid PNA couplings of a known enzyme, millions of slight variations of the known enzyme can be generated automatically. Many of these new variations will be more efficient than the original enzyme at a given task. The variations can be run through an industry standard automated testing and quality assurance process to identify the more efficient enzymes, which are moved into production. The entire development process for a novel enzyme can be accomplished in a period between three and six months, depending upon the complexity of the enzyme.

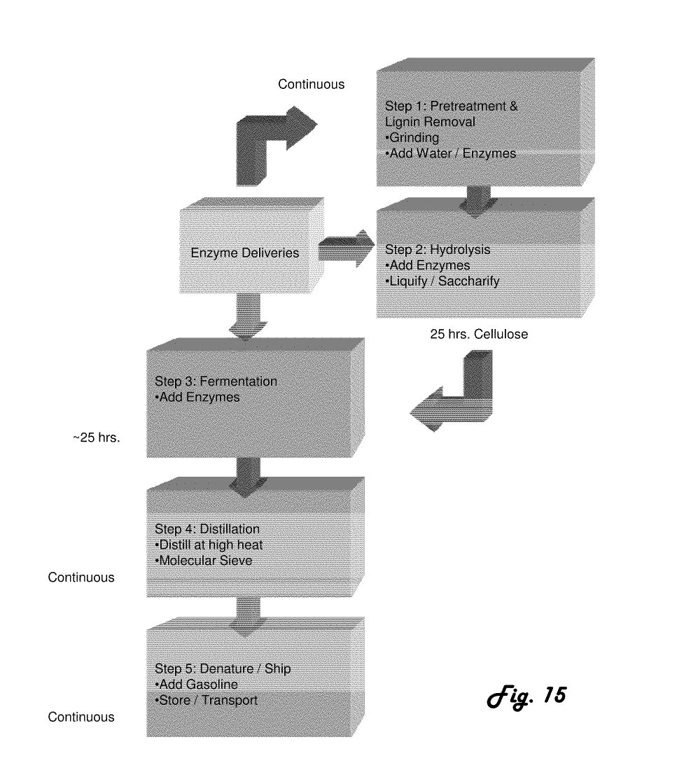

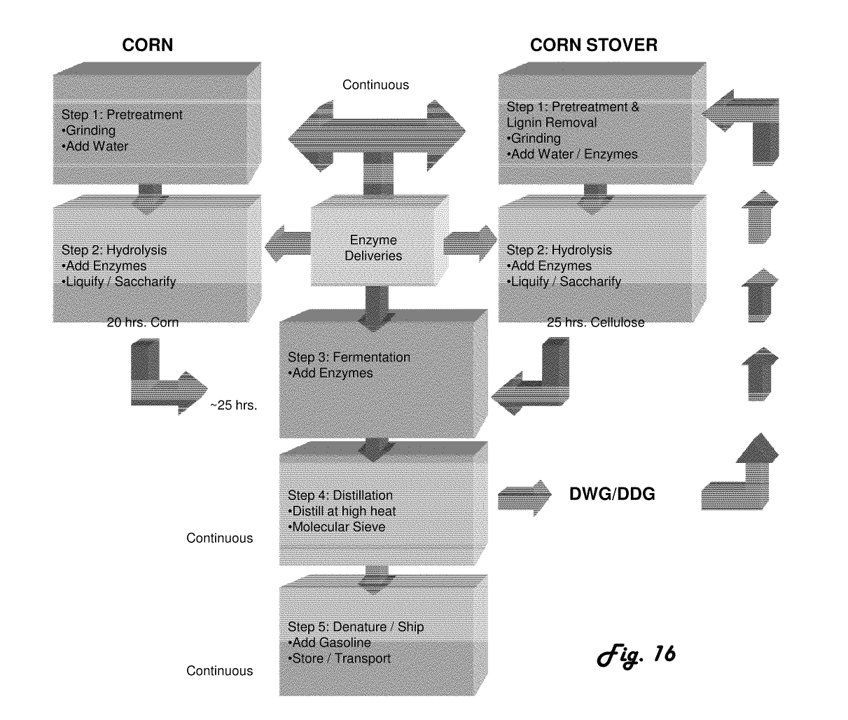

Ethanol Production Example. FIG. 14 is a schematic illustration of an example ethanol production process according to an embodiment of the present invention. Using enzymes produced with the present invention can allow several improvements in the production process, as illustrated in FIG. 15. Reducing the time of cellulosic hydrolysis reduces the number of tanks required for the hydrolysis step, dramatically reducing capital infrastructure costs. Using enzymes for lignin removal also reduces capital cost—there are no special filters or tubing required. Changing to an enzyme based fermentation process reduces operating expenditures in energy, yeasts and chemicals. Improving cellulosic ethanol production enzymes also opens new opportunities to current producers in the corn ethanol market. Current corn ethanol producers can potentially add-on a cellulosic production stream using, for example, corn stover as the supplemental feedstock. Such a process, illustrated in FIG. 16, can be accomplished with minimal additional capital expenditures.

Embodiments of the present invention can also be used, as an example, is in the area of vaccine or drug delivery. Peptide vaccines can be attached to the PNA molecules within the DNA weave, with the weave inverted to present the peptides on the outside of the structure. This can increase the absorption rate within the body to improve effectiveness while lowering the manufacturing cost, providing pharmaceutical companies with hundreds of millions of dollars of annual cost savings.

The present invention has been described as set forth herein. It will be understood that the above description is merely illustrative of the applications of the principles of the present invention, the scope of which is to be determined by the claims viewed in light of the specification. Other variants and modifications of the invention will be apparent to those of skill in the art.

What is claimed is:

1. A method for enabling the connection of additional elements or molecules to a polynucleobase biopolymer weave comprising incorporating additional chemical linkages within the polynucleobase biopolymer weave.

2. A method according to claim 1, wherein at least one additional chemical linkage enables amide coupling.

3. A method according to claim 1, wherein at least one additional chemical linkage enables cycloaddition.

4. A method according to claim 1, wherein at least one additional chemical linkage enables ester coupling.

5. A method according to claim 1, wherein at least one additional chemical linkage enables disulfide linking.

6. A method according to claim 1, wherein the polynucleobase biopolymer weave comprises a nucleic acid.

7. A method according to claim 6, wherein the nucleic acid comprises PNA (peptide nucleic acid).

8. A method according to claim 6, wherein the nucleic acid comprises DNA (deoxyribonucleic acid).

9. A method according to claim 6, wherein the nucleic acid comprises RNA (ribonucleic acid).

10. A method according to claim 6, wherein the nucleic acid comprises LNA (locked nucleic acid).

11. A method according to claim 6, wherein the nucleic acid comprises GNA (glycol nucleic acid).

12. A method according to claim 6, wherein the nucleic acid comprises TNA (threose nucleic acid).

13. A method of generating polynucleobase derivatives, comprising providing an additional chemical linkage aside from the Watson-Crick base pairing link or the backbone link.

14. A method according to claim 13, wherein the polynucleobase comprises thymine.

15. A method according to claim 13, wherein the polynucleobase comprises uracil.

16. A method according to claim 13, wherein the polynucleobase comprises cytosine.

17. A method according to claim 13, wherein the polynucleobase comprises guanine.

18. A method according to claim 13, wherein the polynucleobase comprises adenine.

19. A method according to claim 1, wherein the molecules are placed in relation to each other with tolerances of less than about 10 nanometers.

20. A method of generating a catalyst, comprising using the method of claim 1 to generate a catalyst mimicking known enzymatic transformations but having superior properties to the analogous natural system regarding stability toward pH, temperature, the presence of additives known to accelerate catalysis, or a combination thereof.

21. A method of generating an electrical circuit, comprising using the method of claim 1 to produce a 3D addressable assemblies of polynucleobases with feature size less than about 1/16 of the feature size currently amenable to scaled fabrication by photolithographic techniques.
